(19) United States
(12) Patent Application Publication
(10) Pub. No.: US 2016/0143740 A1
(43) Pub. Date: May 26, 2016

(54) SIZE MEASURING TOOL FOR ARTIFICIAL ANNULUS

(71) Applicants: Hiroyuki TSUKUI, Tokyo (JP); Young-Kwang PARK, Tokyo (JP); Naohiko KANEMITSU, Tokyo (JP); EBM CORPORATION, Tokyo (JP)

(72) Inventors: Hiroyuki TSUKUI, Tokyo (JP); Young-Kwang PARK, Tokyo (JP); Naohiko KANEMITSU, Tokyo (JP)

(21) Appl. No.: 14/776,376

(22) PCT Filed: Mar. 7, 2014
(86) PCT No.: PCT/JP2014/055978

§ 371 Date: Feb. 8, 2016

(30) Foreign Application Priority Data
Mar. 15, 2013 (JP) 2013-054023

(51) Int. Cl. A61F 2/24

(52) U.S. Cl. CPC A61F 2/2496

(57) ABSTRACT

To provide an artificial annulus sizing tool capable of determining a suitable size for an artificial annulus to be implanted in a patient.

[Solution] An artificial annulus sizing tool comprising an annular core material and a cover material covering the core material to a predetermined thickness, the cover material portion being attached to the annulus using suturing thread for implanting artificial annuli, and the part of the cover material engaged with the suturing thread being broken after a suitable artificial annulus size is determined for the annulus, allowing for removal from the suturing thread.

### FIELD OF THE INVENTION

The present invention relates to an artificial annulus sizing tool used to determine the size of an artificial annulus when implanting an artificial-annulus-forming member as treatment for insufficiency of the mitral valve situated between the left atrium and the left ventriculum.

### BACKGROUND OF THE INVENTION

A backflow-preventing valve referred to as the mitral valve is located between the left atrium and the left ventriculum of the heart. The mitral valve opens simultaneously with the contraction of the left atrium, sending blood into the left ventriculum, and closes simultaneously with the contraction of the left ventriculum, serving to prevent the flow of blood back to the left atrium.

FIG. 1 depicts the mitral valve. The mitral valve comprises a roughly ellipsoid annulus 1 situated between the left atrium and the left ventriculum, and two leaflets, an anterior leaflet 2 and a posterior leaflet 3, that extend from the annulus 1 toward the left ventriculum. During diastole, negative pressure forms in the interior of the left ventriculum, causing the anterior leaflet 2 and posterior leaflet 3 to open so that blood flows from the left atrium to the left ventriculum; during systole, positive pressure forms in the interior of the left ventriculum, causing the anterior leaflet 2 and the posterior leaflet 3 to coapt and preventing the flow of blood from the left ventriculum back to the left atrium.

However, the annulus 1 of the mitral valve can, through various causes, expand or become enlarged, with the result that the anterior leaflet 2 and posterior leaflet 3 do not coapt, allowing slight amounts of blood to flow back even during systole. This condition is known as mitral valve insufficiency, and is typically treated via mitral valve annuloplasty.

In mitral valve annuloplasty, an artificial annulus is sutured to the annulus in order to correct the expanded annulus 1 back to its original size. FIG. 2 is a schematic illustration of a process of fitting an artificial annulus 4 to the expanded annulus 1 and suturing the former to the latter. Because the diameter of the annulus 1 varies between patients, it is vital to select an artificial annulus of a suitable size for the artificial annulus 4. Therefore, a special measure (annulus sizing tool) 5 known as a “sizer” is conventionally placed against the afflicted area to measure the diameter of the annulus, as shown in FIG. 3. The surface area of the anterior leaflet 2 and the commissural distance H between the anterior leaflet 2 and the posterior leaflet 3, as shown in FIG. 1, is used as the standard for selecting the size of the artificial annulus 4 during this process.

In order to measure these two items (surface area and commissural distance), a conventional sizer 5 comprises two notches 6, 7, as shown in FIG. 3, that are matched with the commissural distance. A flat plate section corresponds to the surface area of the anterior leaflet, and the sizer 5 exhibiting the nearest dimensions and area yields the size of the optimal artificial annulus 4 for the patient.

However, selection standards may vary depending upon the condition suffered by the patient; for example, a downsizing approach is effective in selecting a suitable artificial annulus size for patients suffering functional mitral valve insufficiency. Meanwhile, it is recommended to select the larger size when selecting from two sizes for patients exhibiting dystrophic valve disease. As can be seen, then, there is no unified standard for selection.

In addition, mitral valve annuloplasty is a procedure performed under extracorporeal circulation using a cardiopulmonary bypass; thus, during the procedure, there is no blood in the heart, and the mitral valve maintains a shape not seen in natural physiological conditions. Therefore, it is difficult to reliably determine a suitable size for an artificial annulus when using a sizer such as described above during the procedure.

If, by chance, an artificial annulus not matching the diameter of the mitral valve of the patient is selected, there is a risk of blood flowing back through the mitral valve after the procedure, leading to complications. Moreover, if a flaw in the repair of the mitral valve is discovered following the procedure, the artificial annulus must be swapped out or replaced with an artificial valve; however, as the insurance costs of artificial annuli can run into the hundreds of thousands of yen, replacing annuli during surgery presents an extremely weighty economic burden, which creates another difficulty.

One strategy for solving these problems is the invention disclosed in patent document 1.

The invention disclosed in patent document 1 is a pseudo-artificial annulus that has a size different from that of an ordinary sizer and a shape corresponding to that of an artificial annulus, with engaging projections for engaging artificial annulus suturing thread being provided on the surface thereof, and annulus suturing thread being wrapped around the engaging projections in order to engage there with and anchor the sizer to the annulus. In accordance with this invention, this pseudo-artificial annulus can be used to perform a valve function evaluation test using physiological saline before the artificial annulus is attached, allowing for reliable evaluation of the size of the artificial annulus to be attached. In addition, the document indicates that, when removing the pseudo-annulus from the annulus, the artificial annulus suturing thread need only be removed from the engaging projections, simplifying the process.

However, in the invention disclosed in patent document 1, it is necessary to wrap suturing thread around the projections on the pseudo-annulus; this wrapping action is not present in ordinary clinical techniques. For example, the thread tends to slacken during wrapping, and it is difficult to sense the state of the thread when adjusting pressure following wrapping. Therefore, this pseudo-annulus cannot be considered as being sutured in a manner similar to that actually used in clinical practice in a state similar to an actual artificial annulus. Therefore, using a pseudo-annulus of this sort cannot be considered to yield reliable valve function evaluation.

In addition, because the projections have outer diameters of only a few millimeters, the suturing thread exhibits a high level of curvature (bending) when being wrapped. The projections are rigid, and present the risk of damage to the thread. In addition, the positions of the projections are fixed. Therefore, it is impossible to alter the suturing position or number of sutures to fit the situation.

Patent document 1: Unexamined Japanese Patent Application Publication 2008-104472

### SUMMARY OF THE INVENTION

The present convention was conceived in view of these circumstances, and has an object of providing a sizer that is capable of evaluating post-operative mitral valve leakage and valve behavior even when the mitral valve is in a non-physiological state. In particular, an object of the present invention is to provide a sizer that allows the evaluation described above to be performed swiftly and without placing burdens upon patients undergoing extracorporeal circulation, and allows mitral valve behavior to be evaluated via a method that is inexpensive and can easily be adopted at hospitals.

In order to attain the objects described above, a main aspect of the present invention is an artificial annulus sizing tool comprising an annular core material and a cover material covering the core material to a predetermined thickness, the cover material portion being attached to the annulus using suturing thread for implanting artificial annuli, and the part of the cover material engaged with the suturing thread being broken after a suitable artificial annulus size is determined for the annulus, allowing for removal from the suturing thread, the cover material being formed so that the break strength thereof with respect to the suturing thread is such that a physician can perform said breakage by applying force of a level that will not damage the annular tissue and so that the material will not break when a force is applied that is less than the force of a level that will not damage the annular tissue applied by the physician during said breakage.

In the embodiments of the present invention, the following aspects are essential.

1) Having a Suitable Break Strength

The artificial annulus sizing tool of the present invention will not function if the break strength thereof is too high or too low.

A suitable range for break strength is a level of strength such that failure will not occur when the cover material is sutured with the suturing thread and the physician performs sizing, and also a level allowing for easy breakage when the artificial annulus sizing tool of the present invention is held in one hand and the suturing threads are pulled with the other hand.

A level of strength allowing for easy breakage is a level that will not burden the annular tissue of the patient during the act of breaking, or that will not cause a physician to be concerned about the risk of such.

2) No Off-Gassing

The artificial annulus sizing tool of the present invention is used within the human body, especially the interior cavities of the heart. When breaking the cover material following sizing, it is imperative that no fragments of the cover material separate from the base material and remain within the interior cavities of the patient's heart. Therefore, the cover material of the present invention must be of a material and structure that permits only the suturing threads to release and produces no other fragments or gas.

For example, in an embodiment of the present invention, break strength can be adjusted so as to satisfy the conditions described above by adding oil to a silicone resin constituting a main component of the cover material during manufacture.

In accordance with one embodiment of the present invention, the break strength is set so as to be in a range of 0.2 N to 4.5 N when breaking using #20 suture thread.

In accordance with another embodiment, the artificial annulus sizing tool configured as described above is modeled after the external shape and size of the artificial annulus being sized, and several types of tools are prepared according to various shapes and sizes.

In accordance with yet another embodiment, the core material has a letter-C shape. Alternatively, the core material may have an endless ring shape.

In accordance with yet another embodiment, the core material is formed by injection molding a resin material.

In accordance with yet another embodiment, the cover material comprises a reinforcing material and a cushioning material that envelops the cover material and covers the core material, and the suturing thread is passed through the cover material so as to penetrate the resin material and the reinforcing material.

In accordance with yet another embodiment, the cushioning material is silicone resin.

In accordance with yet another embodiment of the artificial annulus sizing tool, the cover material has a specific level of viscoelasticity so as only to be broken by the suturing thread without shedding any material.

In accordance with the present invention, the following effects can be obtained.

(1) Because the artificial annulus sizing tool of the present invention is directly sewn in place using thread used to suture the artificial annulus in place, and can be immediately removed, no special preparations are necessary, and the tool can easily be incorporated into the surgical theater.
(2) Following suturing to the mitral valve, the area can be flushed using physiological saline to visually evaluate leakage and changes in the shape of the mitral valve, allowing for more reliable selection of an artificial annulus of optimal size.
(3) Only a very short time, on the order of a few minutes, is necessary from suturing until evaluation, and there is little effect upon operating time, thus also reducing the burden placed upon the patient.

Other features and noteworthy effects of the present invention will be apparent to a person skilled in the art from the embodiment described in the “Best mode for embodying the invention” section and from the drawings.

### BRIEF DESCRIPTION OF THE DRAWINGS

FIG. 1 is a schematic illustration of the mitral valve.

FIG. 2 is a schematic illustration of an artificial annulus being attached.

FIG. 3 is a schematic illustration of a method of using a conventional example of a sizer.

FIG. 4 is a plan view of a sizer according to an embodiment of the present invention.

FIG. 5 is a longitudinal cross-sectional view of the embodiment along line A-A in FIG. 4.

FIG. 6 is a longitudinal cross-sectional view of the embodiment along line B-B in FIG. 4.

FIG. 7 is a magnified schematic illustration of the cross-sectional surface of the embodiment.

FIG. 8 is a schematic illustration of a good example of suturing thread insertion in the embodiment.

FIG. 9 is a schematic illustration of a poor example of suturing thread insertion in the embodiment.

FIG. 10 is a flow chart for a method of manufacturing a sizer according to the embodiment.

FIG. 11 is a schematic illustration of a fiber material being cut according to the embodiment.

FIG. 12 is a schematic illustration of a core material being bent according to the embodiment.

FIG. 13 is a schematic illustration of molding using a mold according to the embodiment.

FIG. 14 is a schematic illustration of a molded article being bent and shaped according to the embodiment.

FIG. 15 is a schematic illustration of a process of attaching and removing a sizer in the embodiment.

FIG. 16 is a schematic illustration of a process of attaching and removing a sizer in the embodiment.

FIG. 17 is a schematic illustration of a process of attaching and removing a sizer in the embodiment.

FIG. 18 is a schematic illustration of a process of attaching and removing a sizer in the embodiment.

FIG. 19 is a plan view of a sizer according to another embodiment of the present invention.

FIG. 20 is a plan view of a sizer according to another embodiment of the present invention.

FIG. 21 is a graph of testing results.

FIG. 22 is a graph of testing results.

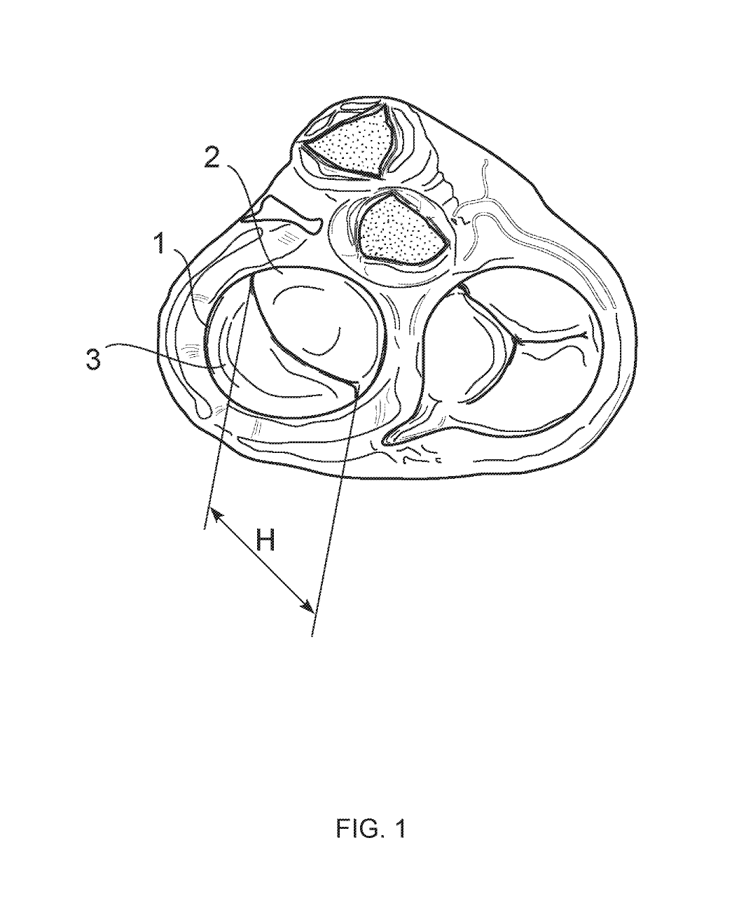

### DETAILED DESCRIPTION OF THE INVENTION

An embodiment of the present invention will now be described with reference to the attached drawings. In the following description, the artificial annulus sizing tool of the present invention will be referred to as a “sizer”.

(Configuration of Sizer)

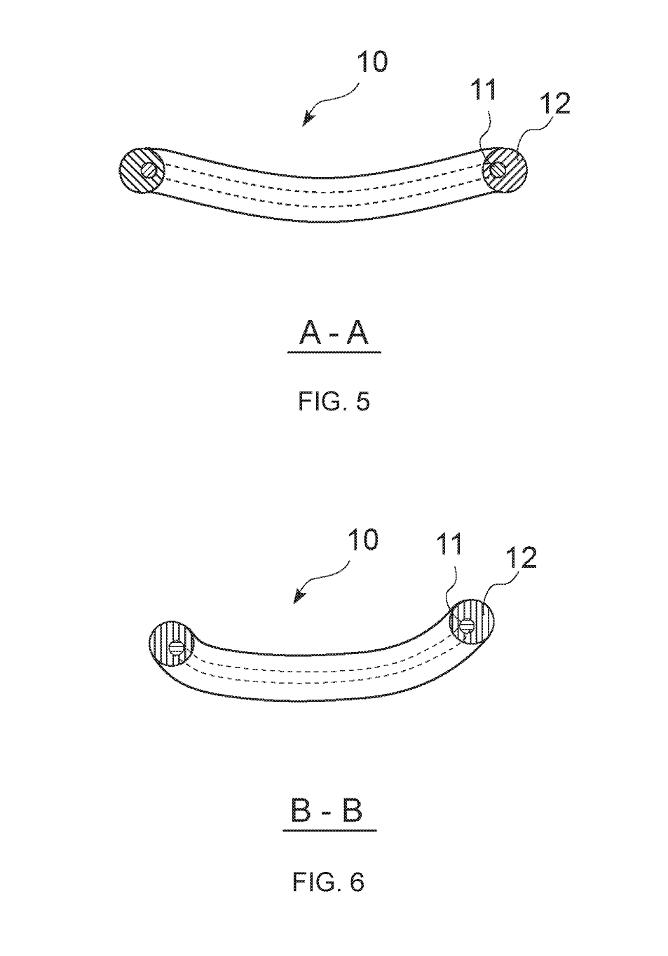

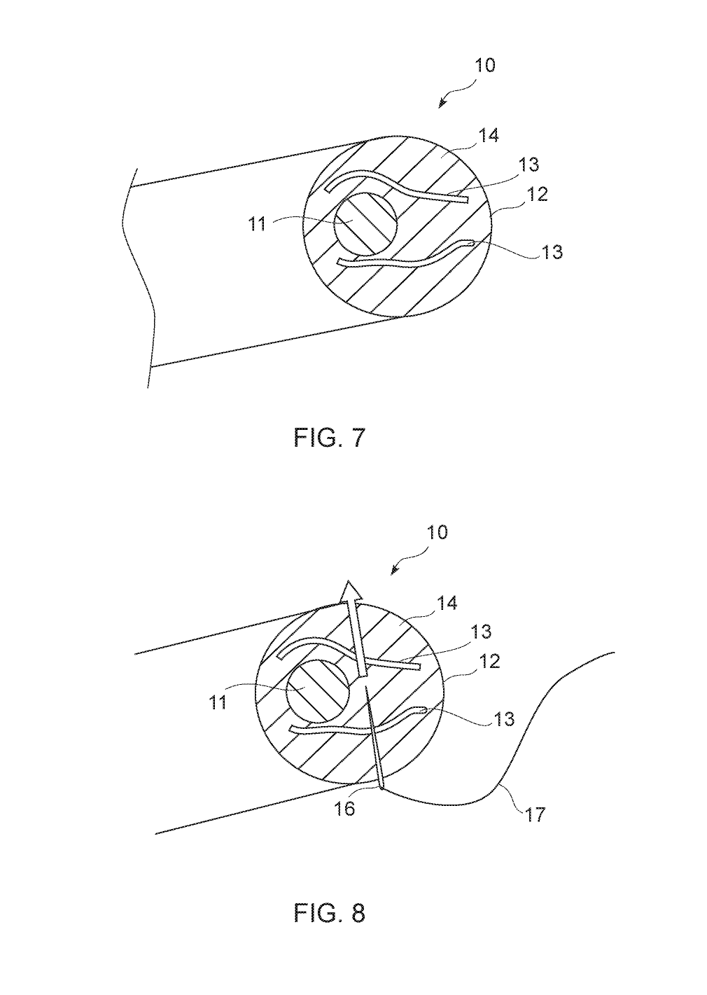

FIG. 4 is a plan view of a sizer 10 according to the present embodiment, FIG. 5 is a cross-sectional view along line A-A (major axis) thereof, and FIG. 6 is a longitudinal cross-sectional view along line B-B (minor axis) thereof. FIG. 7 shows a magnified view of the longitudinal cross section of the sizer 10.

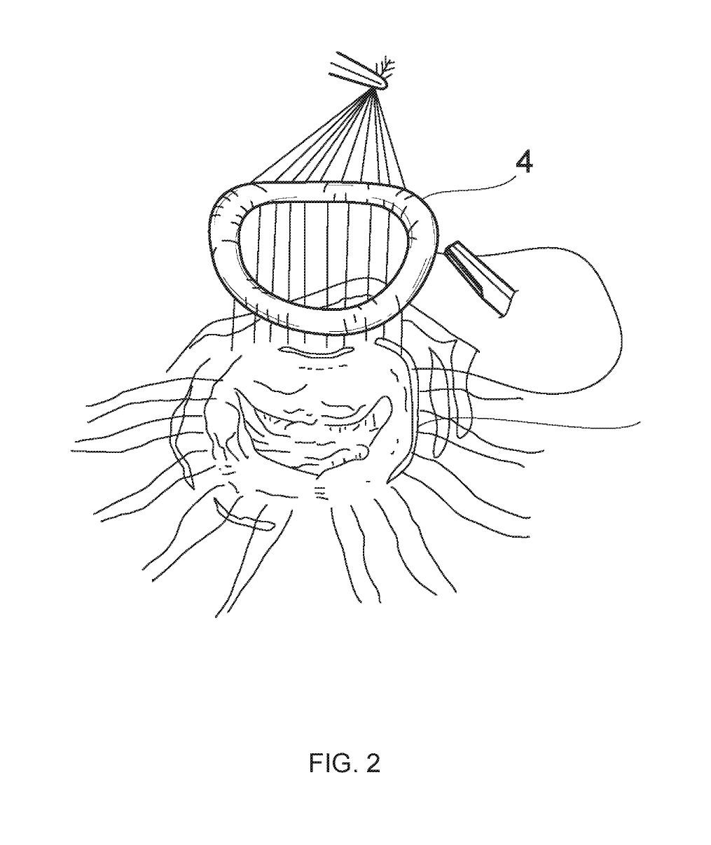

As shown in FIGS. 5 to 7, the sizer 10 comprises a core material 11 and a cover material 12 covering the core material 11. As shown in magnified view in FIG. 7, the cover material 12 is constituted by a fiber material 13 disposed around the core material 11, and a section of silicone rubber 14 that envelops the fiber material 13 and seals the core material 11. The sizer 10 has an endless ring shape (roughly identical to that of the artificial annulus 4; see FIG. 2), as shown in FIG. 4, and is formed in an overall curved shape having a concave central section, as shown in the longitudinal cross-sectional views of FIGS. 5 and 6.

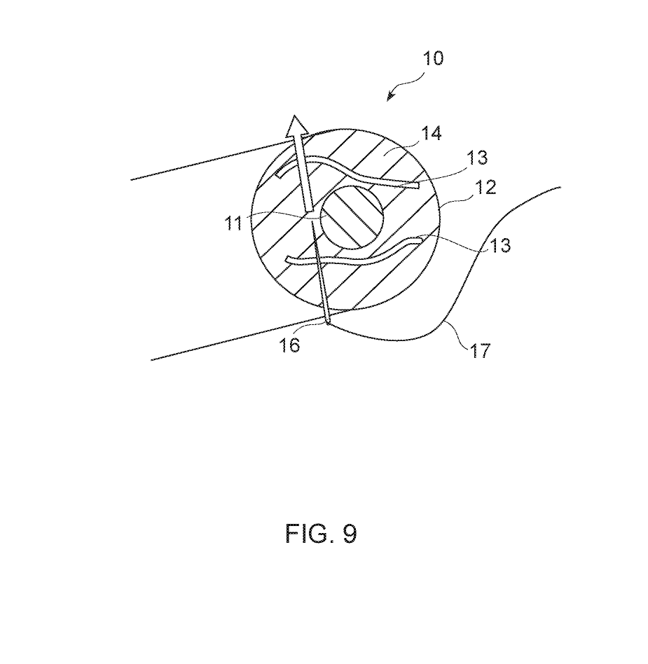

The core material 11 is, for example, a stainless steel wire having a diameter of 1.2 mm, and has a specific level of rigidity, with the result that the core serves to maintain the shape of the sizer 10 during the procedure. As shown in FIG. 7, the core material 11 is disposed within the cover material 10 so as to be offset toward the center of the sizer 10. This arrangement prevents the needle from entangling the core material 11 when the sizer 10 is being sutured to the annulus. Specifically, when using a needle 16 to pass thread 17 through the cover material 12 of the sizer 10, the core material 11 will rarely be entangled if disposed in an offset position as shown in FIG. 8, whereas there is a possibility of the needle 16 passing to the inside of the core material 11 if the core material 11 is disposed at a central position as shown in FIG. 9. Such entanglement of the core material 10 by the needle 16 must be avoided, as this will make it impossible to remove the sizer 10 from the thread 17 later.

The cover material 12 (fiber material 13 and silicone rubber 14) serves as a body through for passing the thread 17 (needle 16) through the sizer 10. Of the various elements constituting the cover material 12, the fiber material 13 is constituted by a polyurethane elastomer. The polyurethane elastomer is soft and stretchable, but is durable against being broken by the thread 17, allowing the elastomer to serve as a reinforcing material that improves the ability of the thread 17 to hold the sizer 10 in place while maintaining the flexibility of the sizer 10. Meanwhile, the silicone rubber 14 serves as a shock-absorbing material that, along with the fiber material, yields a specific level of break strength, as will be discussed hereafter.

Multiple sizers 10 of different sizes having the shape described above are provided, and sizers of different sizes can be exchanged, as appropriate, to identify a single optimal size for the annulus of the patient.

In the present embodiment, for example, nine sizers are provided in which the average of the outer diameter and the inner diameter along the major axis varies in 2 mm intervals from 24 mm to 38 mm. The diameter of the cross section of the sizers varies from 3.5 to 5 mm as the sizers increase in size.

(Method of Manufacturing Sizer)

Next, the configuration of the sizer 10 according to the present embodiment will be described in further detail by describing a method of manufacturing the sizer 10.

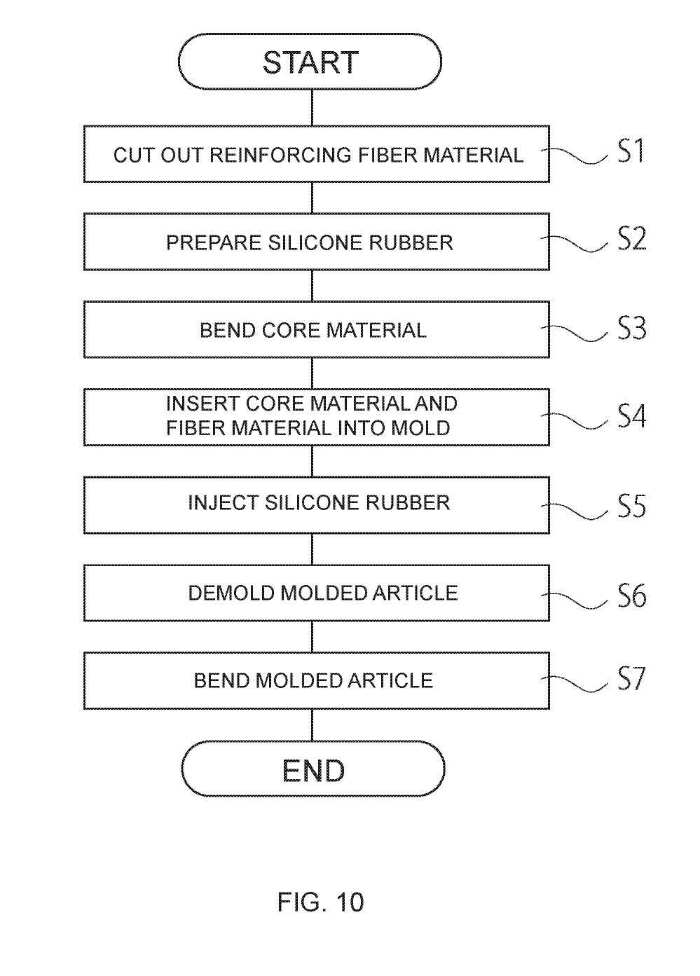

FIG. 10 is a flow chart for a method of manufacturing a sizer. The labels S1 to S7 in the drawing correspond to steps S1 to S7 described hereafter.

### (1) Step S1: Cutting the Reinforcing Fiber Material

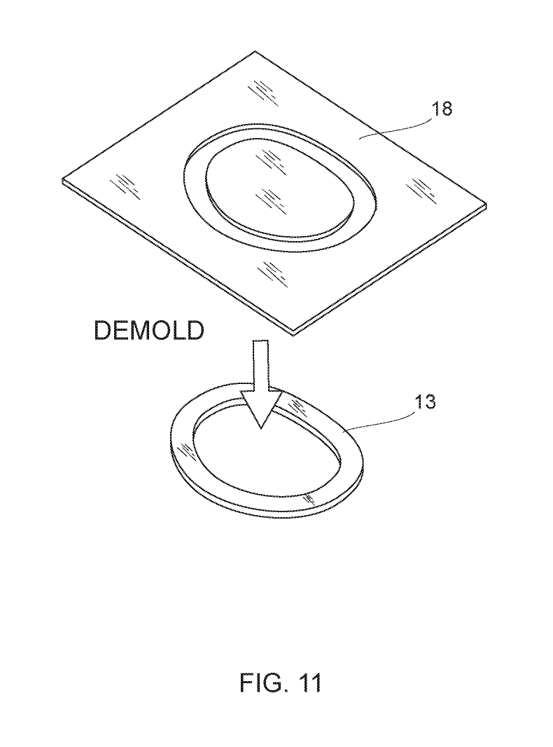

In step S1, the polyurethane elastomer fiber material 13 is cut out using a laser cutter into the same shape as the sizer 10 for implantation into the sizer as a reinforcing material. FIG. 11 shows the fiber material 13 having been cut from a polyurethane elastomer fiber material sheet 18.

### (2) Preparation of Silicone Rubber

In step S2, a specific proportion of silicone rubber 14 constituting the greater part of the cover material 12 is prepared. It is important to bear the following points in mind and select optimal materials when deciding upon the materials and composition for the cover material 12 (including the fiber material 13 and silicone rubber 14)

Specifically, the artificial annulus sizing tool of the present invention is used within the human body, especially the interior cavities of the heart. When breaking the cover material 12 following sizing, it is imperative that no fragments of the cover material 12 separate from the base material and remain within the interior cavities of the patient's heart. Therefore, the cover material of the present invention must be of a material that permits only the suturing threads to release and produces no other fragments or gas.

Therefore, in the present embodiment, viscoelasticity is imparted and break strength is adjusted to a level satisfying the conditions described above by adding oil to the silicone rubber 14 constituting a main component of the cover material 12 during manufacture.

### (3) Bending of Core Material

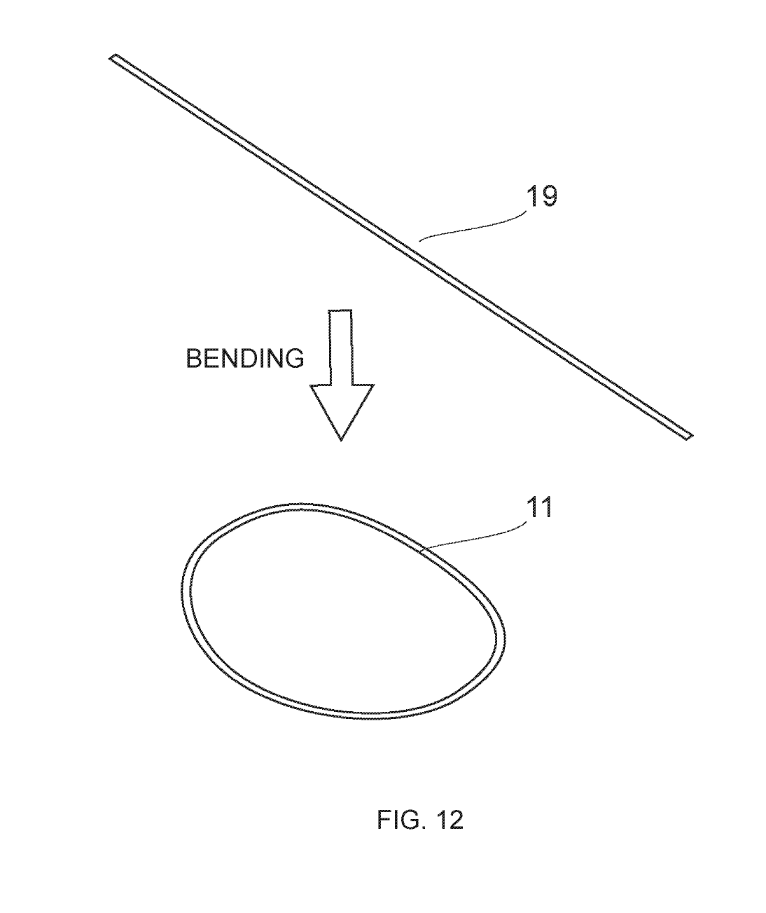

In step S3, a stainless steel wire 19 such as shown in FIG. 12(a) is bent using a wire bender into the same shape as the sizer, as shown in FIG. 12(b).

(4) Insertion of Core Material and Fiber Material into Mold

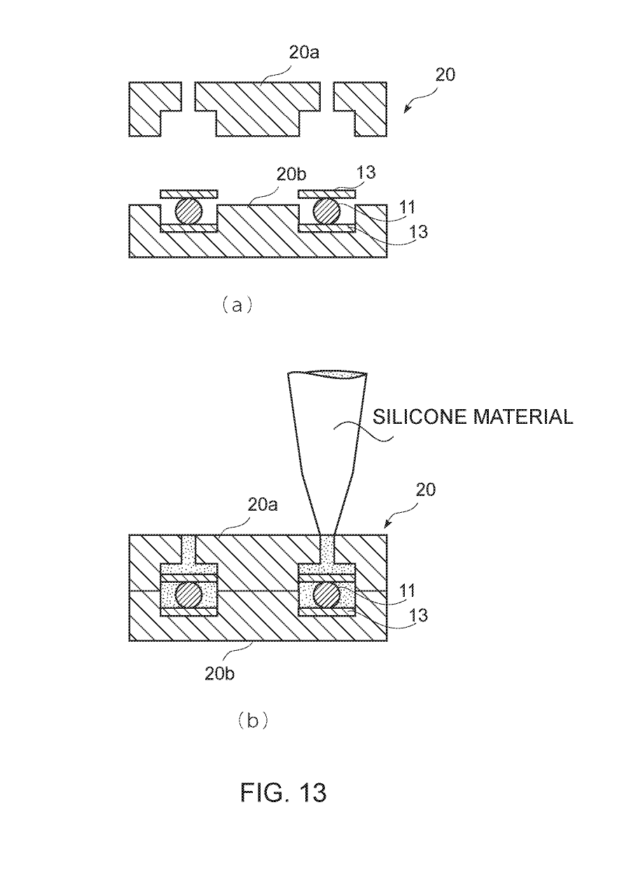

In step S4, the core material 11 and fiber material 13 prepared in steps S1 and S3 are disposed in a lower mold 20a of a mold 20 in the order fiber material 13, core material 11, fiber material 13, as shown in FIG. 13.

### (5) Injection of Silicone Rubber

In step S5, upper and lower molds 20a, 20b of the mold 20 are closed together, and silicone rubber 14 is injected through a resin injection inlet 21 in the upper mold 20b. Subsequently, the mold is heated for one hour at a predetermined curing temperature to cure the silicone rubber 14.

The silicone rubber 14 is a thermoset resin that cures when heated, affecting manufacturing efficiency. However, the rubber has the property of exhibiting increased physical properties values for elasticity and brittleness following curing if the heating temperature is higher than a certain level.

For this reason, manufacturing at optimum efficiency at the desired physical properties is possible by heating the rubber at a suitable temperature.

It is also possible to sterilize the surface of the sizer 10 through this heating. Specifically, the present invention is used within the human body, especially the endocardial spaces, albeit for short periods of time; thus, the surface of the article must be sterilized.

(6) Demolding from Mold

In step S6, the mold 20 is cooled once heating is complete, and the molded article is removed.

### (7) Bending of Molded Article

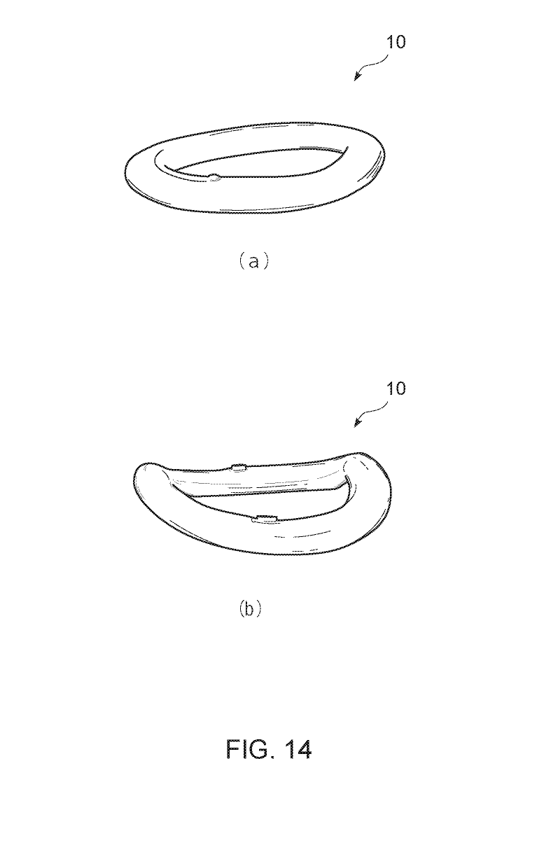

In step S7, the molded article is bent using a specific jig to deform the article into a saddle-like shape as shown in FIG. 14. This completes the sizer 10 of the present embodiment.

(Method of Using Sizer)

Next, a method of using the sizer 10 of the present embodiment will be described.

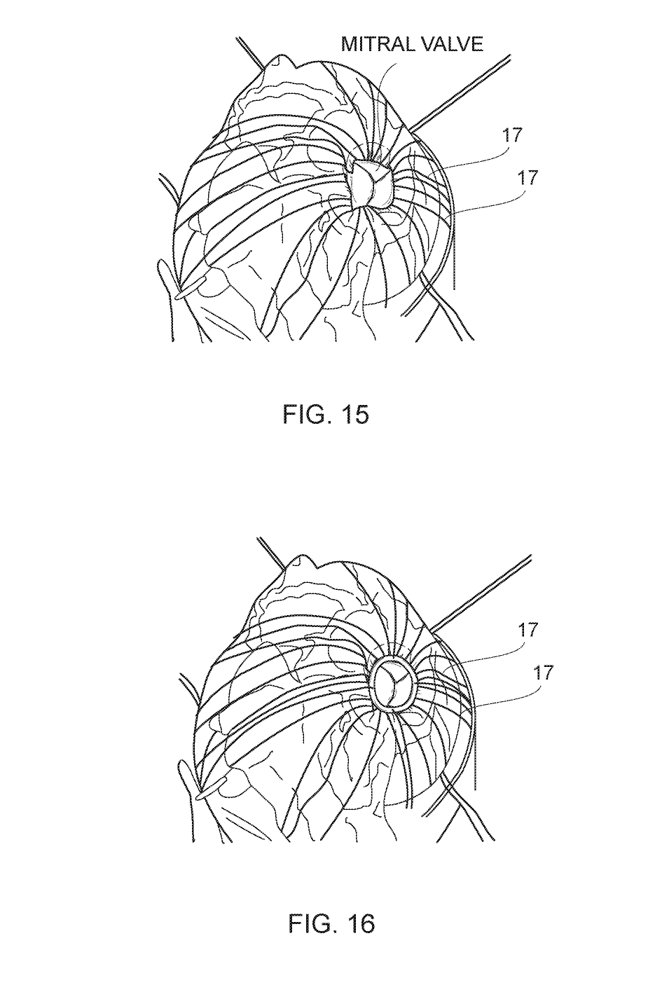

In a mitral valve annuloplasty using an artificial annulus, the periphery is first sutured in place using thread 17, as shown in FIG. 15, thereby exposing the mitral valve and establishing a field of view.

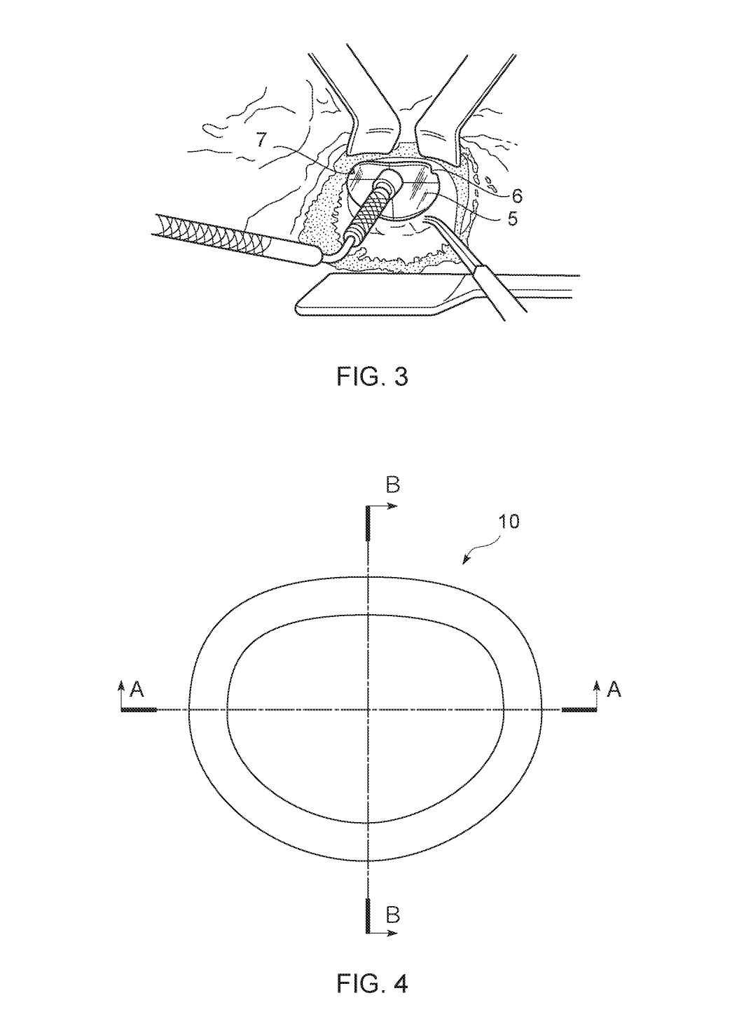

In conventional procedures, a sizer 5 of the same shape as the artificial annulus is then placed against the mitral valve as shown in FIG. 3 to determine an optimal size; in the present embodiment, however, the sizer 10 described above is used to size the artificial annulus instead of the sizer shown in FIG. 3.

About four stitches of thread 17 for suturing the artificial annulus in place are used to actually attach the sizer 10 of the present invention to the annulus of the mitral valve, as shown in FIG. 16. During this process, the thread 17 is passed through the cover material 12 so that the needle 16 passes to the outside of the core material 10, as shown in FIG. 8. During this process, it is vital that the needle 16 not passed to the inside of the core material 11, as shown in FIG. 9; the eccentric disposition of the core material 11 in the present embodiment allows the chances of this happening to be reduced.

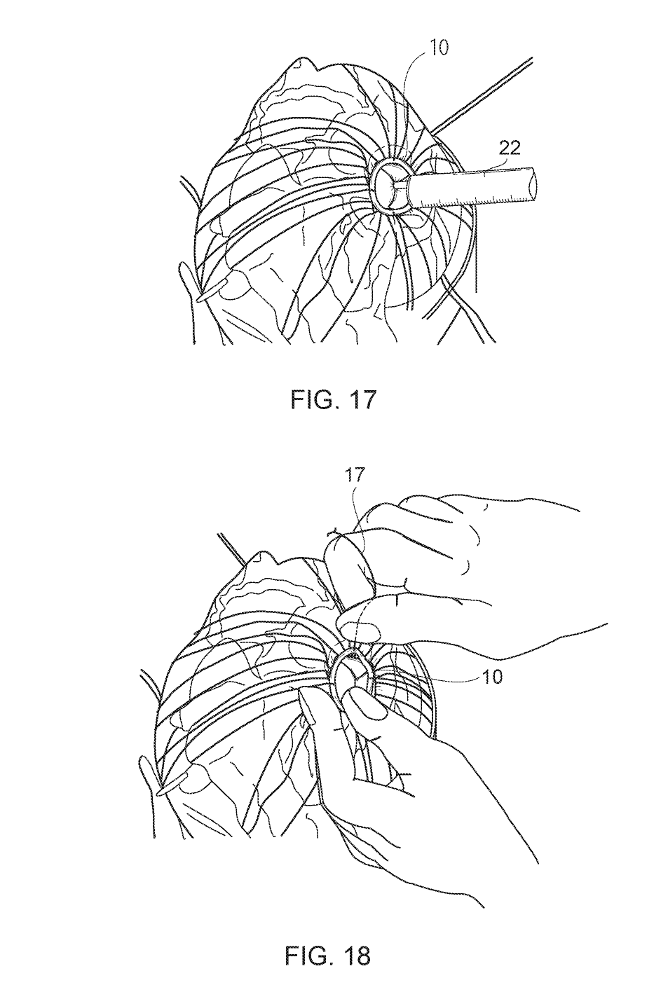

Next, physiological saline is injected using a syringe 22, as shown in FIG. 17, and valve behavior and water leakage are evaluated. Once evaluation is complete, the threads 17 are grasped with the fingers and used to break the cover material 12 of the size of 10, as shown in FIG. 18, and the sizer 10 is removed from the threads 17. If it is determined, as the result of the evaluation, that the size is unsuitable, the process returns to the procedure shown in FIG. 16, and a sizer 10 of a different size is reattached and the behavior of the valve is re-evaluated.

Once a sizer 10 of an optimal size has been identified, an artificial annulus of the same size as the sizer 10 is selected and attached according to a procedure similar to that used in ordinary operations.

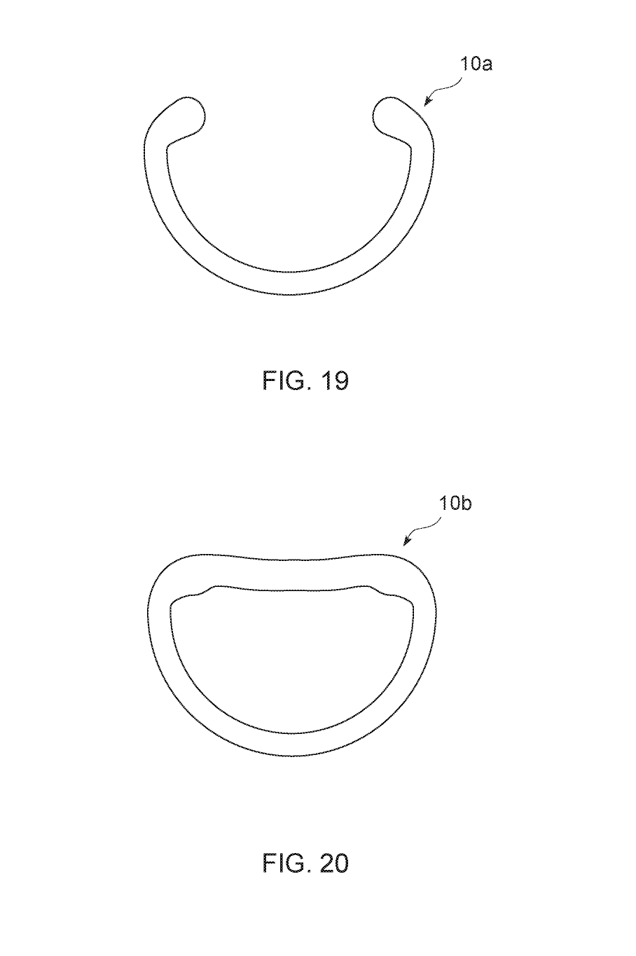

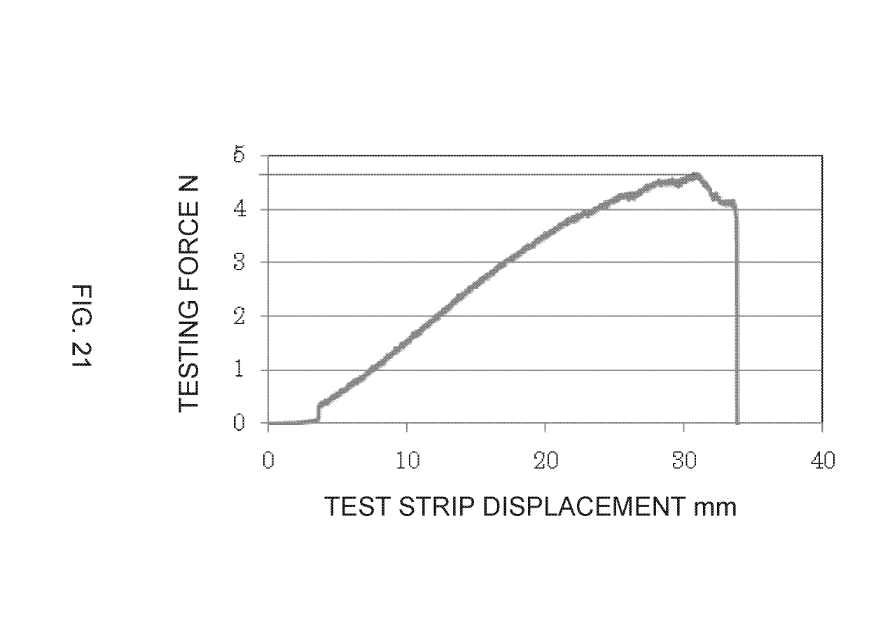

Therefore, the size of 10 of the present invention is preferably prepared so as to match commercially available artificial annuli of various shapes actually used in clinical practice. In other words, the sizer of the present invention is not limited to the shapes shown in FIGS. 4 to 7, and may have other shapes. For example, the sizer may have shapes such as those shown in FIGS. 19 and 20 (letter-C shape, deformed ring-shape).

(Cover Material Break Strength Test Examples)

Next, testing performed in order to identify optimal specifications for the cover material used in the sizer of the present embodiment will be described.

1. Purpose of Testing

Because the sizer of the present invention is used for immediate manual evaluation during a mitral valve annuloplasty, the sizer must simultaneously be strong enough to withstand the process of being attached to the mitral valve and brittle enough to be manually broken and removed by the physician once sizing is complete. Defining “strength” as break strength, the following test was performed in order to quantify a range for break strength that is applicable for the sizer.

Specifically, the sizer of the present invention will not function if the break strength thereof is too high or too low.

A suitable range for break strength is a level of strength such that failure will not occur when the cover material is sutured with the suturing thread and the physician performs sizing, and also a level allowing for easy breakage when the sizer of the present invention is held in one hand and the suturing threads are pulled with the other hand.

A level of strength allowing for easy breakage is a level that will not burden the annular tissue of the patient during the act of breaking, or that will not cause a physician to be concerned about the risk of such.

2. Testing Method

Two types of testing were performed: a qualitative evaluation test performed by physicians, and a quantitative breaking test. First, physicians sutured test strips formed from various materials capable of constituting the cover material of the sizer, and qualitatively evaluated whether or not the materials exhibited a level of strength suitable for a sizer material. Next, a breaking test was performed to quantify the strength of the test strips and attain an applicable range of strengths for the sizer.

3. Qualitative Testing Performed by Physicians

### (1) Purpose of Testing

To select a material strength allowing for application as a sizer cover material via qualitative evaluation performed by physicians.

### (2) Test Sample Specifications

Square-cut strips were prepared for the test strips of the test. A commercially available rubber sheet that was comparatively hard and believed to have a high level of strength was used for the test strip used to determine an upper limit. Softened silicone rubber obtained by adding oil to a base material of molding silicone rubber was used for the test strip used to obtain a lower limit. Specific specifications for the test strips are shown in tables 1 and 2.

TABLE 1

| Test | | Silicone rubber/oil weight ratio | | |
|---|---|---|---|---|
| No. | Test strip name | KE1310ST | KF-96-50CS | Oil content |
| 1 | ST20 | 10 | 20 | 47.6% |
| 2 | ST30 | 10 | 30 | 64.5% |
| 3 | ST32.5 | 10 | 32.5 | 73.2% |
| 4 | ST35 | 10 | 35 | 76.1% |
| 5 | ST37.5 | 10 | 37.5 | 77.3% |
| 6 | ST40 | 10 | 40 | 78.4% |
| 7 | ST42.5 | 10 | 42.5 | 79.4% |
| 8 | ST45 | 10 | 45 | 80.4% |
| 9 | ST47.5 | 10 | 47.5 | 81.2% |
| 10 | ST50 | 10 | 50 | 82.0% |
| 11 | ST52.5 | 10 | 52.5 | 82.7% |
| 12 | ST55 | 10 | 55 | 83.3% |

TABLE 2

| Test No. | Name | Thickness (mm) |
|---|---|---|
| 1 | Black cell sponge | 3 |
| 2 | Plain rubber | 3 |
| 3 | Neocell rubber | 1.5 |
| 4 | EVA sheet | 2 |
| 5 | Black rubber sheet | 0.5 |
| 6 | Black rubber sheet | 1 |
| 7 | Black rubber sheet | 2 |
| 8 | Black rubber sheet | 3 |

Grid lines were drawn at 1 mm intervals to a distance of 3 mm from the ends of the testing strips in order to serve as stitching guides for the needle during the test.

### (3) Testing Method

In the test, the suture thread was passed through the test strip in order to place the same load thereupon as placed upon the sizer, and the durability of the test strip with respect to the load was qualitatively evaluated. The test was performed according to the following procedure.

3-1. The test strip was clamped in place using chucks.
3-2. Suturing thread used in mitral valve annuloplasties was passed through the test strip from the rear. The guideline for the distance from the ends of the test strip to the stitching position was 2 mm, a value obtained from previous extermination.
3-3. After a certain length of thread had passed through the test strip, the thread was used to break the test strip.

### (4) Evaluation Method

The test strips were evaluated by soliciting comments from the physicians regarding the following two points.

4-1. Whether or not the material could be used as material for a sizer
4-2. Additional comments regarding strength and behavior

### (5) Results

Evaluation results for the rubber sheets and silicone sheets are shown in tables 3 and 4. The physician comments indicated that, of the rubber sheets used to obtain an upper limit, the black cell sponge and the Neocell rubber were of usable strength. Regarding the silicone sheets used to obtain a lower limit, comments indicated that silicone rubber up to S400 was usable, but that oiled rubber test strips of S425 or higher broke during testing, making them too brittle for application to sizers.

TABLE 3

| | | Stitching | | |
|---|---|---|---|---|
| | | distance | | |
| No. | Type | (mm) | Evaluation | Physician comments |
| 1 | S200 | 3.0 | OK | Usable line. |
| 2 | S300 | 3.0 | OK | Durability not bad. Usable line. |
| 3 | S325 | 3.0 | Fail | Strength slightly low |
| 4 | S350 | 4.0 | OK | Usable line. |
| 5 | S375 | 3.0 | OK | Slightly weak, but not bad. Usable |
| | | | | line. |
| 6 | S400 | 3.0 | OK | Usable line. |
| 7 | S425 | 3.0 | Fail | The needle holes expanded when |
| | | | | the threads were moved. |
| 8 | S450 | 4.0 | Fail | Poor strength; could not withstand |
| | | | | four stitches. |
| 9 | S475 | 2.0 | Fail | Soft, weak. |
| 10 | S500 | 3.0 | Fail | The needle holes expanded when |
| | | | | the threads were moved. |
| 11 | S525 | 3.0 | Fail | Too weak to use. |
| 12 | S550 | 3.5 | Fail | So soft it shed fragments. |

TABLE 4

| | | | Stitching | | |
|---|---|---|---|---|---|
| | | Thickness | distance | | |
| No. | Name | (mm) | (mm) | Evaluation | Physician comments |
| 1 | Black cell | 3.0 | 3.0 | OK | Just barely OK |
| | sponge | | | | |
| 2 | Plain rubber | 3.0 | 3.0 | Fail | Too sticky; would pull on |
| | | | | | tissue when removed. |
| 3 | Neocell | 1.5 | 3.0 | OK | Hard, but should be usable. |
| | rubber | | | | Acceptable if no other options |
| | | | | | were available. |
| 4 | EVA sheet | 2.0 | 3.0 | Fail | Too hard to use. |
| 5 | Black | 0.5 | 3.0 | Fail | Good strength, but unusable |
| | rubber sheet | | | | due to fragment shedding. |
| 6 | Black | 1.0 | 2.5 | Fail | Showed signs of shedding |
| | rubber sheet | | | | fragments. |
| | | | | | (No comment regarding |
| | | | | | strength) |
| 7 | Black | 2.0 | 2.0 | Fail | Would not break at all. |
| | rubber sheet | | | | |
| 8 | Black | 3.0 | 2.5 | Fail | Would not break at all. |
| | rubber sheet | | | | |

### (6) Summary

An applicable range of break strengths for the sizer was determined via qualitative testing using rubber sheets and silicone sheets.

S400 was closest to the minimum applicable specifications for hardness; evaluation showed that rubber having the oil content of S425 or higher was unusable.

The evaluation indicated that, of the various rubber sheet test strips, black cell sponge and Neocell rubber were closest to the upper limit for strength.

### 4. Break Testing

### (1) Purpose of Testing

This test was a break test performed upon some of the test strips used in the qualitative evaluation. The results obtained from this test were compared with the results of the physician evaluations to set an applicable range of break strength for the sizer.

### (2) Test Sample Specifications

The test strips had the same shape as the test strips used in the qualitative testing (see tables 5, 6).

TABLE 5

| | | | Stitching | Evaluation | |
|---|---|---|---|---|---|
| Test | | | disance | from | |
| No. | Material | Thickness | (mm) | qualitative | Reason for selection |
| 1 | Black cell | 3 | 3 | OK | Rated as maximum value |
| | sponge | | | | during qualitative test. |
| 2 | Neocell | 1.5 | 3 | OK | Rated as maximum value |
| | rubber | | | | during qualitative test. |
| 3 | Plain rubber | 3 | 3 | Fail | Comments indicated that, of |
| | | | | | the various failed test trips, |
| | | | | | this one had a level of |
| | | | | | strength considerably |

TABLE 6

| | | | Evaluation | |
|---|---|---|---|---|
| | | Stitching | from | |
| Test | | distance | qualitative | |
| No. | Name | (mm) | test | Reason for selection |
| 4 | S350 | 4 | OK | Evaluated as having strength near |
| | | | | lower limit in qualitative test. |
| 5 | S375 | 3 | OK | Evaluated as having strength near |
| | | | | lower limit in qualitative test. |
| 6 | S400 | 3 | OK | Evaluated as having strength at |
| | | | | lower limit in qualitative test. |
| 7 | S425 | 3 | Fail | Evaluated as having strength less |
| | | | | than lower limit in qualitative test. |

### (3) Testing Method

The procedure for the break test is described hereafter, and test conditions are shown in table 7. The position at which the suturing thread (in this embodiment, #20) was inserted was set at 3 mm based on the distance at which the needle was actually inserted into the test strips during qualitative testing.

1. Suturing thread inserted into test strip.

2. Test strip and suturing thread chucked on tensile tester. During testing, air chucks were used so that the suturing thread would not slip.

3. Load upon test strip confirmed as being 0[N].

4. Tester operated to determine break strength.

TABLE 7

| Apparatus used | All-purpose tensile tester |
|---|---|
| | AG-X 5 kN (Shimadzu) |
| Test rate | 100 mm/min |
| Suturing thread type | 2-0 ETHIBOND (ETHICON) |
| Stitching distance | 3 mm |
| Exposed test strip length | 10 mm |
| No. of samples | 6 |

### (4) Evaluation Method

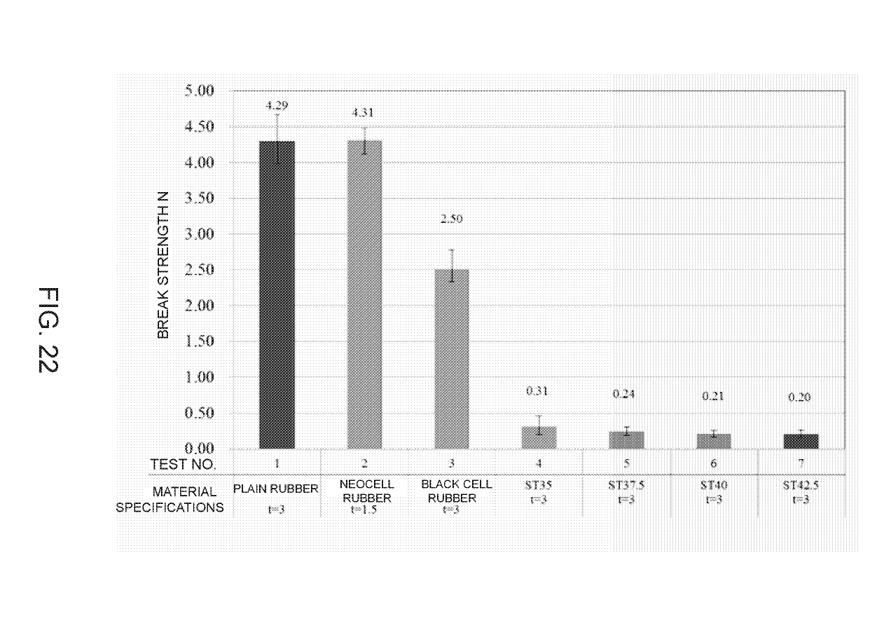

As shown in FIG. 21, the test strips exhibited maximum resistance at the moment at which they broke from the threads. In this test, this maximum value was defined as break strength, and the break strengths of each of the test strips were determined and compared.

### (5) Results

Tensile test results are shown in FIG. 22.

### Upper Limit Setting

Break strengths of 2.5[N] and 4.31[N] were obtained for the black cell rubber and the Neocell rubber, respectively, which were evaluated as having levels of strength just at the upper limit. The average value for plain rubber deemed unusable was 4.29[N], slightly less than that of the Neocell rubber.

However, whereas the Neocell rubber had a maximum value of 4.48[N], the plain rubber had a maximum value of 4.67[N], a value greater than that of the Neocell rubber. If it is necessary to draw a line between usable and unusable for the plain rubber and the Neocell rubber, 4.5[N], a value exceeding the upper limit for the Neocell rubber that can be demonstrated by the plain rubber, is believed to be the upper limit value for break strength.

### Lower Limit Setting

Although no significant difference in break strength could be ascertained between S400 and S425, the minimum value for S350 was 0.20[N], and the minimum value for S375, for which the physician comments indicated uncertainty regarding strength, was 0.19[N]; thus, concerns regarding strength are believed to appear at a lower limit of 0.2[N].

S400 test strips exhibiting a break strength exceeding 0.2[N] and S425 test strips exhibiting a break strength of less than 0.2[N] were obtained at a probability of 50%; it is believed that, if a model in which S400 has a strength of 0.2[N] or greater and a model in which S425 exhibits a strength of less than 0.2[N] is used during qualitative evaluation, the results of the qualitative evaluation and the results of the break test should exhibit the same tendency.

### (6) Summary

Break tests were performed on test strips that exhibited values for break strength near the upper and lower limits in qualitative testing.

The break strengths of the Neocell rubber and the plain rubber were compared, and an upper limit of 4.5[N] was set for the break strength of the sizer.

The break strengths of the S350 and S425 were compared, and a lower limit of 0.2[N] was set for the break strength of the sizer.

As a result of the testing described above, it was determined that a range having a lower limit of 0.2[N] and an upper limit of 4.5[N] was preferable for the strength of the cover material of the sizer.

The present invention is not limited to the embodiment described above, and various modifications may be made thereto to the extent that they do not alter the gist of the invention.

For example, although stainless steel wire is used for the core material 11 in the example described above, a plastic core of identical shape can be manufactured via injection molding. In addition, while the core material 11 is first molded before being covered by the cover material 12 in the embodiment described above, it is also possible to first cover the core material 11, followed by performing bending so as to mold the overall shape.

In addition, the cover material 12 is not limited to the configuration of the example described above. While a fiber material was used in the embodiment described above, it is also acceptable to use only the cushioning material constituted by the silicone rubber 14 or the like as long as the desired break strength is exhibited. Moreover, the cushioning material is not limited to being silicone rubber; various materials, including those used in the test examples described above, can be selected.

The present invention is not limited to the embodiment described above, and various modifications may be made thereto to the extent that they do not alter the gist of the invention.

### REFERENCE NUMBERS

10: Sizer
11: Core material
12: Cover material
13: Fiber material
14: Silicone rubber

1. An artificial annulus sizing tool comprising:
an annular core material and a cover material covering the core material to a predetermined thickness;
the cover material portion being attached to the annulus using suturing thread for implanting artificial annuli, and the part of the cover material engaged with the suturing thread being broken after a suitable artificial annulus size is determined for the annulus, allowing for removal from the suturing thread;
the cover material being formed so that the break strength thereof with respect to the suturing thread is such that a physician can perform said breakage by applying force of a level that will not damage the annular tissue and so that the material will not break when a force is applied that is less than the force of a level that will not damage the annular tissue applied by the physician during said breakage.

2. The artificial annulus sizing tool according to claim 1, wherein:
the break strength is set so as to be in a range of 0.2 N to 4.5 N when breaking using #20 suture thread.

3. The artificial annulus sizing tool according to claim 1, wherein:
the artificial annulus sizing tool is modeled after the external shape and size of the artificial annulus being sized, and several types of tools are prepared according to various shapes and sizes.

4. The artificial annulus sizing tool according to claim 1, wherein:
the core material has a letter-C shape.

5. The artificial annulus sizing tool according to claim 1, wherein:
the core material has an endless ring shape.

6. (canceled)

7. The artificial annulus sizing tool according to claim 1, wherein:
the core material is formed by injection molding a resin material.

8. The artificial annulus sizing tool according to claim 1, wherein:
the cover material comprises a reinforcing material and a cushioning material that envelops the reinforcing material and covers the core material; and
the suturing thread is passed through the cover material so as to penetrate the resin material and the reinforcing material.

9. The artificial annulus sizing tool according to claim 1, wherein:
the cushioning material is silicone resin.

10. The artificial annulus sizing tool according to claim 1, wherein:
the cover material has a specific level of viscoelasticity so as only to be broken by the suturing thread without shedding any material.

11. The artificial annulus sizing tool according to claim 1, wherein:
the core material is eccentrically disposed within the cover material.
